(19) United States
(12) Patent Application Publication
(10) Pub. No.: US 2013/0274788 A1
(43) Pub. Date: Oct. 17, 2013

(54) METHODS AND APPARATUS FOR OCULAR SURGERY

(71) Applicants: Colman Kraff, (US); Terry A. Fuller, (US); THE BOARD OF REGENTS OF THE UNIVERSITY OF TEXAS SYSTEM, Austin, TX (US)

(72) Inventors: Thomas Jennings, Dallas, TX (US); Colman R. Kraff, Chicago, IL (US); Terry A. Fuller, Rydal, PA (US)

(21) Appl. No.: 13/858,657

(22) Filed: Apr. 8, 2013

Related U.S. Application Data
(60) Provisional application No. 61/622,844, filed on Apr. 11, 2012.

(51) Int. Cl. A61B 17/02

(52) U.S. Cl. CPC A61B 17/0231
USPC 606/191

(57) ABSTRACT

Provided are devices and methods for controlling fluid egress from the eye during ocular surgery, including an ocular seal that is signed to fit in an incision in an eye tissue such as the cornea or sclera and includes one or more lumen for passage of instruments into the eye without permitting loss of fluids through the incision. Also provided is a system for cataract surgery including a globe stabilization device, a laser lens removal device, one or more corneal seals, an infusion line, and an anterior chamber pressure monitor.

### CROSS REFERENCE TO RELATED APPLICATIONS

This application claims priority based on U.S. Provisional Application Ser. No. 61/622,844 filed Apr. 11, 2012, which is incorporated herein by reference.

### FIELD OF THE INVENTION

This invention relates generally to apparatus and methods for improved surgical procedures on the eye.

### BACKGROUND OF THE INVENTION

Without limiting the scope of the invention, its background is described in connection with ocular surgery as exemplified by cataract surgery as it is currently practiced. Surgery for the treatment of cataracts has been practiced for well over a thousand years and is now the most common operation in the world. Although removal of the clouded lens from the field of view has been long practiced, replacement with a synthetic intraocular lens (IOL) was not introduced until 1949. The next great advance in cataract surgery was “phacoemulsification”, introduced by Kelman in the 1960s. Phacoemulsification involves separating the lens from the lens capsule such that the lens can move freely and then using ultrasound to emulsify the cataractous lens into small pieces that are removed from the lens capsule by aspiration through a small cannula. Use of phacoemulsification greatly reduced the size of the incision required to remove the diseased lens. After the advent of phacoemulsification, the size of the incision was dictated by the dimensions of the IOL. Modern cataract surgery and lens replacement can be conducted through a main incision of less than about 3 mm using a foldable lens that is inserted into the lens capsule.

More recent cataract surgical technologies are being developed that replace ultrasound for entire removal of the lens. One such system utilizes a water jet-based technology for removing the damaged lens. In phacoemulsification or other lens emulsification procedures, a volume of fluid is introduced into the lens capsule and removed by irrigation thus carrying out pieces of the emulsified lens. Ideally the volume of fluid entering the eye is balanced by the volume leaving the eye. In practice however, an undesirable excess of fluid may leak out of the one or more corneal surgical incisions created and utilized during many cataract procedures. Incisional fluid leakage, as defined by the difference between the total volume of irrigation fluid and the volume aspired by the phacoemulsification machine, can be considerable.

Changes in anterior chamber volume causes the anterior chamber structures to move thus vastly increasing the risk of complications. Such complications include rupture of the posterior aspect of the lens capsule (capsular rupture), which results in vitreous entering into the anterior chamber. Vitreous loss may result in post-operative wound leakage, endophthalmitis due to a vitreous wick, vitreous traction, cystoid macular edema or retinal detachment, and having the lens nucleus drop into the vitreous. All these complications can cause severe decreased post-operative vision.

Presently, for some aspects of the cataract surgery procedure, leakage may be controlled in part by filling the anterior chamber with a viscoelastic fluid (such as for example Amvisc® or Viscoat®, Alcon Laboratories) to help the anterior chamber retain its shape and prevent leakage. The viscoelastic material must then be removed at the completion of surgery. For other aspects of the cataract surgery procedure, large amounts of fluid are infused into the eye to attempt compensation for wound leak. Up to 250 ml of fluid can be infused during a case whereas the anterior chamber and posterior chamber volumes are each 0.5 ml. Large infusions cause corneal endothelial cell loss, can result in corneal edema), may contribute to vitreous syneresis (liquefaction of the vitreous—which can lead to post-operative posterior vitreous detachments, retinal tears and detachment) and fluid penetrating the zonules and bowing the posterior capsule forward with concomitant risk of posterior capsule rupture.

From the foregoing it is apparent that there is a need in the art for methods and apparatus for minimizing leak of fluids and minimizing the complications listed above. Also needed are methods and apparatus that permit automation of the cataract removal process thus making the procedure more reliable and readily available.

### BRIEF SUMMARY OF THE INVENTION

The present invention is directed to improved methods and apparatus for eye surgery including a corneal seal that controls loss of fluid out of the eye during cataract procedures. The devices provided herein have application in cataract surgery but also in other types of intraocular surgery including without limitation retino-vitreal surgeries.

In one embodiment an ocular seal is provided that includes a flexible bladder or ring having an inner circumferential lip and an outer circumferential lip separated by a circumferential groove that are together dimensioned and adapted to sealably fit into a surgical incision in an ocular wall. The bladder or ring further includes at least one lumen adapted to conform to the contours of at least one instrument passed through the lumen into the eye while limiting egress of fluids from the eye. In certain embodiments the bladder or ring is prefilled while in other embodiments that bladder or ring is fillable through a port. In certain embodiments the ocular seal is supplied with an attached reservoir adapted to supply a fluid or gas to the bladder or ring.

In certain embodiments, the ocular seal is provided with a pressure detector adapted to monitor fluid pressure within the eye. The seal may further include an infusion line mounted through a lumen in the seal.

Also provided is a system for cataract surgery including one or more of a globe stabilization device, a lens removal device, one or more corneal seals, an infusion line, and an anterior chamber pressure monitor. In one embodiment, the lens removal device is a laser fiberoptic lens ablation probe that includes a suction conduit, a linear fiber optic wave guide and an angled laser delivery tip that directs laser energy at an angle from the linear fiber optic wave guide.

### BRIEF DESCRIPTION OF THE DRAWINGS

For a more complete understanding of the present invention, including features and advantages, reference is now made to the detailed description of the invention along with the accompanying figures:

FIG. 1A provides a side view illustrating relevant structures of the eye during cataract surgery. FIG. 1B represents a frontal view illustrating relevant structures of the eye during cataract surgery.

FIG. 2A illustrates a three dimensional perspective illustrating certain relevant structures of the eye during cataract surgery. FIG. 2B depicts the leakage holes created in a corneal incision when an instrument is passed through the incision.

FIGS. 3A and B further depict the leakage holes created in a corneal incision when an instrument is passed through the incision.

FIGS. 4A and B illustrate an embodiment of an ocular seal as disclosed herein. FIG. 4C represents cross sectional views through the embodiment of FIG. 4B. FIGS. 4A and 4C depict an embodiment including an optional integral irrigation line.

FIGS. 5A-C depict side views of an alternative embodiment of an ocular seal as disclosed herein when an instrument is passed through the seal.

FIGS. 6A-C depict frontal views of an embodiment of an ocular seal disclosed herein when an instrument is passed through the seal. FIG. 6D represents an embodiment having more than one opening for passage of instruments or fluid conducts.

FIG. 7A depicts a system for cataract surgery including a globe stabilization device, a lens removal device, one or more corneal seals, an infusion line, and anterior chamber pressure monitoring. FIG. 7B depicts one embodiment of a laser probe having an angled laser delivery tip. FIG. 7C depicts another embodiment of a laser probe having an angled laser delivery tip and a combined prism/lens.

### DETAILED DESCRIPTION OF THE INVENTION

Cataract surgery is a very common surgical procedure. With the aging population, the need for cataract surgery throughout the Western and the third world is immense. World-wide over 12 million people are blind from cataracts. Improved methods and apparatus for cataract surgery are continuing world-wide needs. Provided herein are solutions for controlling loss of fluid out of the eye during eye surgery including cataract procedures, which has heretofore been an unmet need.

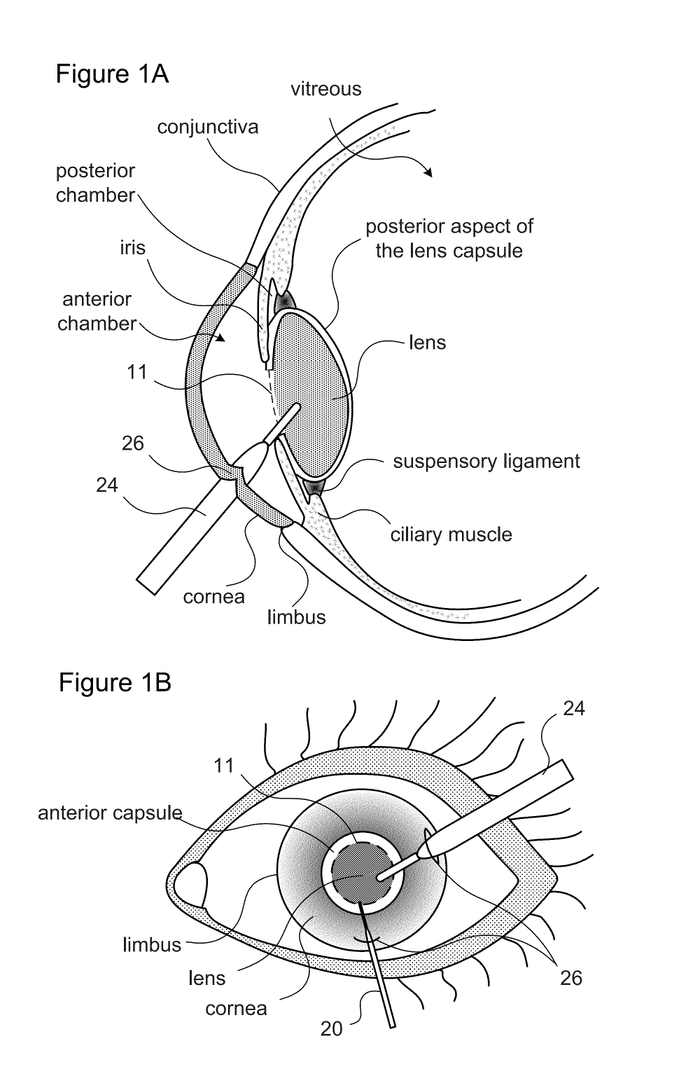

Currently one commonly practiced method of cataract surgery is an extracapsular procedure in which the lens is removed such as by phacoemulsion while leaving at least the posterior aspect of the lens capsule (posterior capsule) in place. Certain of these structures of the eye relevant to the present disclosure are shown in FIG. 1A. As further depicted in FIGS. 1A and 1B, extracapsular cataract surgery as it is typically practiced first involves creation of one or more small straight incision(s) 26 in the cornea. Where several incisions are utilized, there will typically be a small incision for passage of a small instrument 20 that aids in stabilization of structures in the eye and a larger incision for introduction of a lens ablation device 24, such as for example a phacoemulsion wand or laser. If desired a controlled incision may be made such with devices such as those disclosed in Becton Dickinson U.S. Pat. No. 6,371,966.

A capsulotomy is then performed to remove a window of tissue on the anterior aspect of the lens capsule (anterior capsule). The capsulotomy may be performed by controlled tearing or by capsulorhexis, which is a form of capsulotomy involving a controlled excision of a small circular portion 11 of the anterior capsule (dashed lines in FIGS. 1A and B show portion removed), such as by a rotating blade or electric wire.

Next the lens is moved to encourage free rotation within the capsule and the lens ablation device is introduce to begin pulverizing and extracting the lens. In cases where the lens is hardened and does, or is expected to, resist phacoemulsion, a much larger incision must be made to extract the whole lens. After the old lens is removed, a silicone, acrylic or polymethyl methacrylate (PMMA) IOL is implanted in essentially the same location as the original lens.

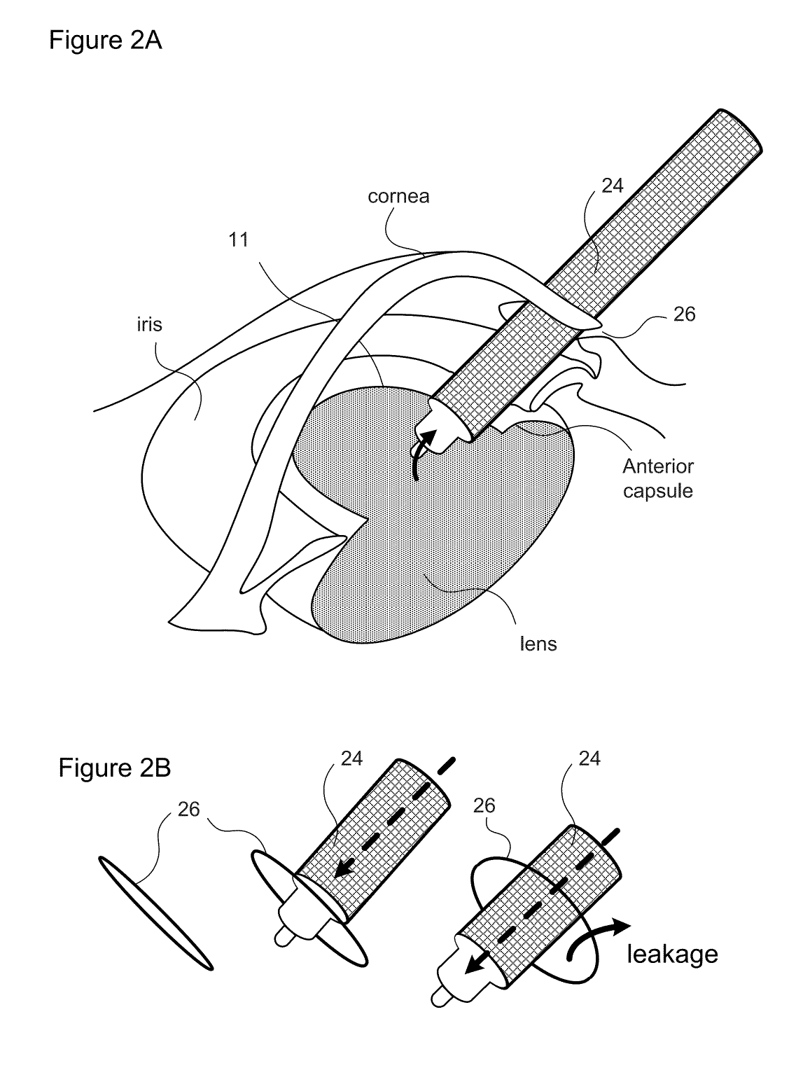

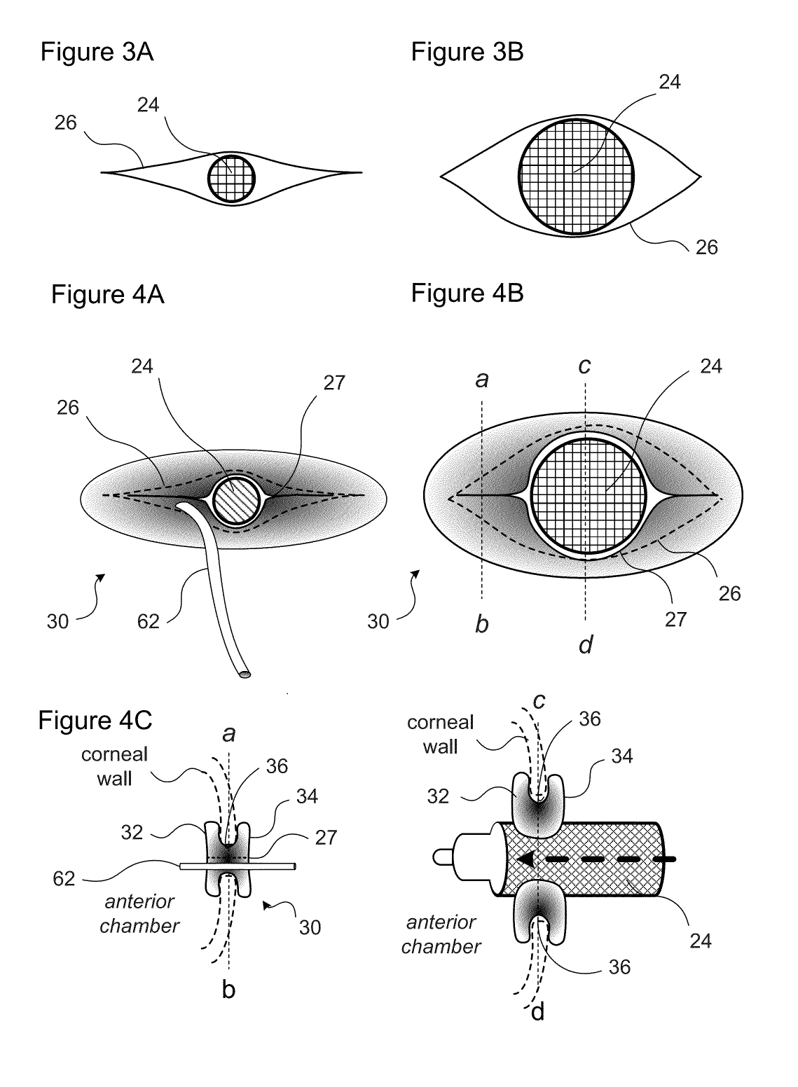

With currently available modalities, and as depicted in FIGS. 2A and B, the mismatch between the outlines of linear surgical incision 26 and the cross-sectional circular shape of surgical instruments 24 opens the incision(s) and allows for leakage out of the anterior chamber. This phenomenon is further depicted in FIGS. 3A and 3B where it is readily appreciated that considerable leakage holes are created once the full outer aspect of an inserted instrument 24 is passed through linear incision 26.

The present inventors appreciated that if the anterior chamber is made to be an essentially closed system during the cataract surgical procedure such that the chamber volume is stable and thus the positions of the ocular structures are known and predictable, the ease of surgery would be increased and the complication rate would decrease. In one embodiment disclosed herein, a closed system is created by placement of a seal 30 that has an outer shape that may resemble a curved rhomboid and one or more openings or lumens located in the seal and dimensioned to drape around instruments inserted through the lumen(s) as depicted in FIGS. 4A and B. Seal 30 straddles the incision 26 and allows instruments 24 to be passed through the more central opening(s). In one embodiment seal 30 is made of a flexible membrane such as medical grade silicon that is prefilled, or filled during surgery, with a gas or fluid. As used herein the term “prefilled” includes a flexible solid but pliable membrane. The size of seal 30 is selected based on the size of instruments that will be passed through it and is adapted and dimensioned to conform to the wound, keep the internal circular hole closed off, and accommodate the insertion of a surgical instrument while conforming to the contours of the instrument to prevent leakage of fluid through the seal. As depicted in FIGS. 4A and 4C, in one embodiment the seal includes an integral irrigation port or line 62.

FIG. 4C shows two cross sections through the embodiment of FIG. 4B. In the cross section through a to b, shown prior to insertion of an instrument, seal 30 has been inserted into an incision in the cornea and the seal is held in place by an inner lip 32 and outer lip 34 having a groove 36 there between that engages the edges of the incised corneal walls. As depicted in the cross section through c to d, as instrument 24 is advanced through incision 26, the opening in seal 30 is expanded and the flexible membrane drapes around the side of the instrument.

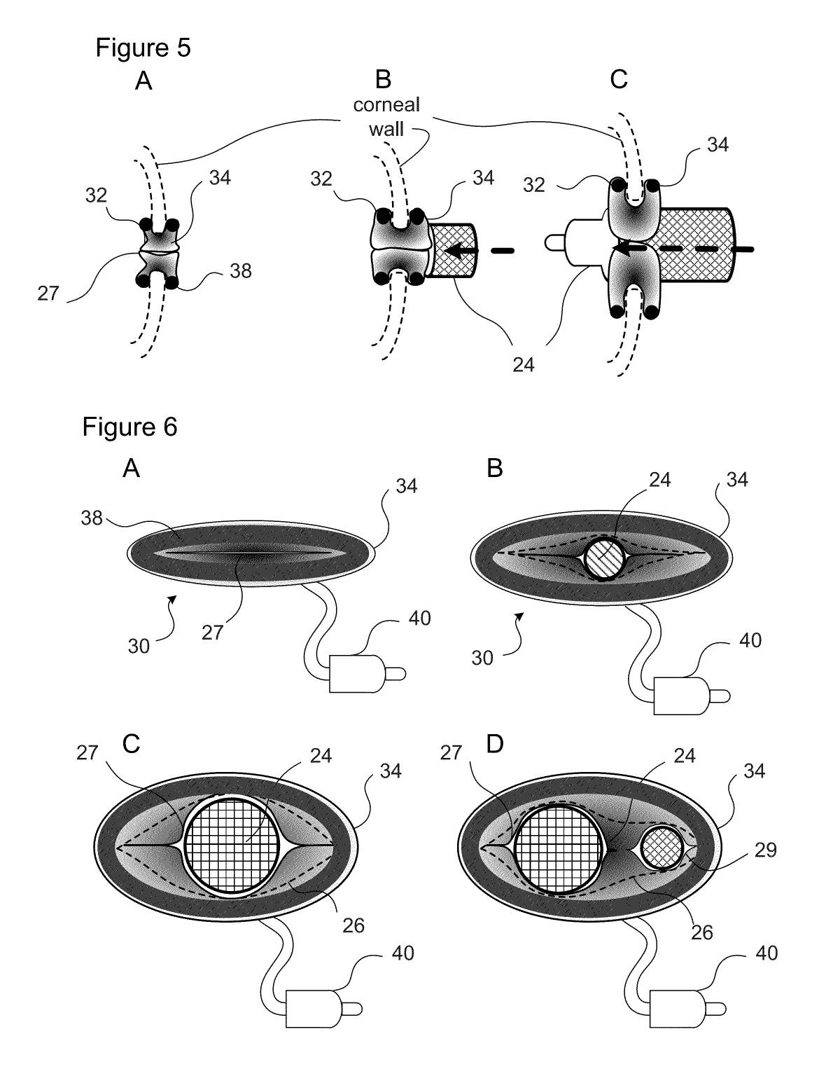

In the alternative embodiment depicted in FIGS. 5 and 6, the seal includes a pair of rings 38 that are pliable but have sufficient rigidity to maintain an outer shape of the seal and hug the opening in the eye surface. In the embodiment of FIG. 5, the seal is shown in side view prefilled to an under pressured level that does not fully distend the fill capacity of the seal. Thus, the seal can be considered somewhat flaccid as supplied and when first inserted. As shown in FIGS. 5B and C, insertion of an instrument 24, causes the under pressured filling to redistribute away from the top and bottom of the incision with movement of the filling to the sides of the seal thus distending those aspects of the seal that allow it to conform to the sides of the inserted instrument and preventing the egress of fluid from the anterior chamber.

FIGS. 6A-C depict several front views of ocular seal 30 as instrument 24 progresses through opening 27 until the fullest outer diameter of instrument 24 enters the seal. The embodiments of FIGS. 6A-D are shown with included rings 38. FIGS. 6A-D also include an alternative filling option where the volume of the seal can modulated through external bladder 40, which may be designed for manual operation or may include a pressure regulating spring that is released once the seal is firmly inserted and seated in incision 26. Thenceforth, the spring maintains a desired fluid or air pressure in the seal 30 but is sufficiently loose to allow the seal filling to move back into bladder 40 when an instrument 24 is passed through the seal thereby displacing a volume of its contents into the bladder. FIG. 6D shows an alternative embodiment wherein the seal 30 includes a further opening 29 that may be used for passage of a further instrument or a fluid conduit such as an infusion line and/or pressure monitor.

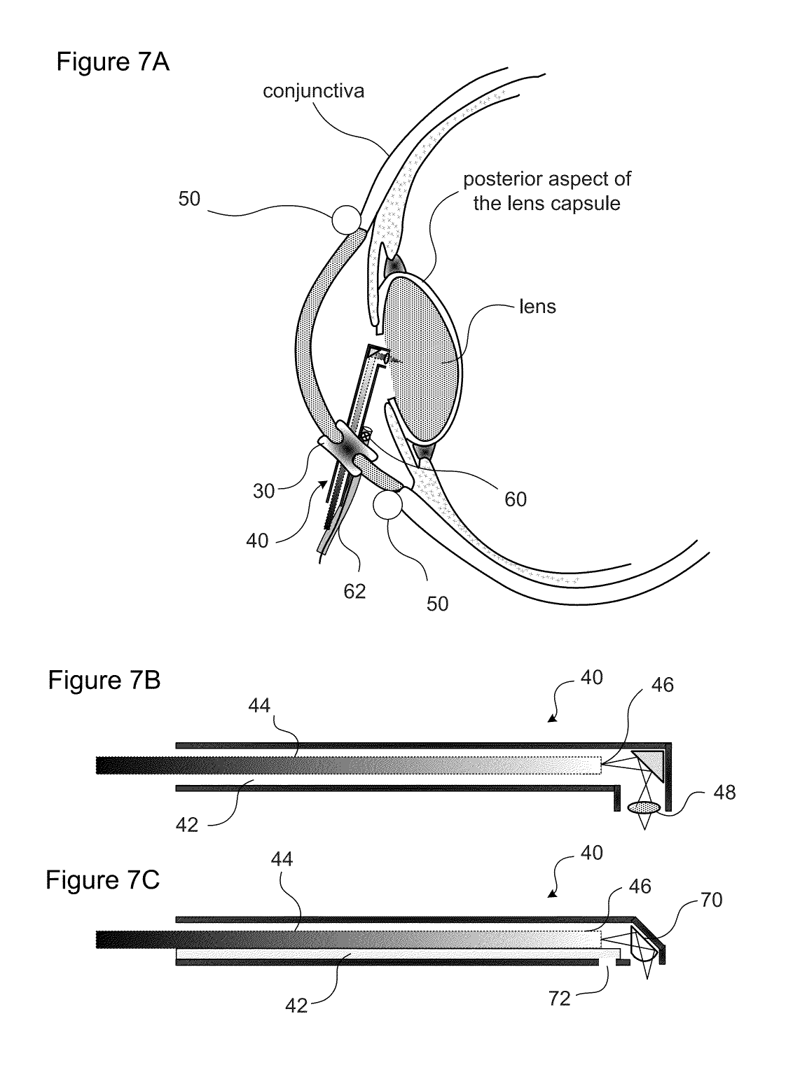

FIG. 7A depicts another embodiment of a system for cataract removal. The depicted system includes a globe stabilization device, a lens removal device, one or more corneal seals, an infusion line, and an anterior chamber pressure monitor. However, it is understood that in practice only certain of the depicted elements might be employed together in a given procedure. In the depicted embodiment, a closed system of fluid in the anterior chamber is provided by a seal 30 inserted into one or more incisions in the cornea. An infusion line 62 is inserted through a seal 30 into the anterior chamber or is integral to the seal. A pressure monitor 60 is also introduced through a seal 30 or is provided as integral to the seal. In one embodiment the rate of infusion through line 62 is set to equal an amount of fluid egress through an aspiration port such as that utilized as part of a phacoemulsification device or the depicted laser lens removal apparatus. Alternatively, the infusion rate may be controlled utilizing a pressure monitor, which may be attached to the cataract removal instrumentation or may be separately inserted. The pressure monitor increases or decreases an infusion rate delivered by a pump (not shown) when the monitor detects a decrease or increase in anterior chamber pressure. The infusion rate may also be increased when an instrument visualizing the anterior chamber detects chamber shallowing.

As further depicted in the system of FIG. 7A, the globe is stabilized by a vacuum suction device, such as for example a limbal suction ring 50 that circumferentially hugs the limbus except at the area of surgical incision. Anterior chamber volume and globe stabilization help ensure that lens removal can be conducted safely without risking posterior capsule rupture. The system depicted in FIG. 7A further depicts use of a femtosecond laser fiberoptic 40 for lens fracturing and aspiration. In one automated embodiment, a set of anterior ocular coherence tomography or anterior segment ultrasound devices are employed that are adapted and dimensioned for placement around the limbus to create a 3-dimensional image of the anterior segment and in particular the boundary of the lens and the exact position of the surgical instrument, during ocular surgery. In certain embodiments the imaging devices are adapted to provide a surgeon alert system where an alarm sounds if an instrument comes too close to the lens capsule or other ocular structures.

As further shown in FIG. 7B, the depicted femtosecond laser fiberoptic lens ablation instrument includes a prism or mirror 46 on the distal end or tip so that laser energy is directed at an angle of about 45° to about 135° to a linear fiber optic wave guide or fiberoptic axis running through the body of the instrument. In the depicted embodiment the angle is about 90° to the linear fiber optic wave guide. In certain embodiments such as that depicted in FIG. 7C, an integrated prism and focusing lens 70 is utilized and has certain benefits. It reduces the number of surfaces femtosecond laser 44 energy needs to traverse and is thus more efficient and at the same time reduces the optical surface area thus reducing debris build up on the optics. It is less expensive to manufacture and can be configured to a very small diameter. In certain embodiments, the angled laser fiberoptic lens ablation instrument is first used to perform an anterior capsulorhexus and then used to remove the central core of lens tissue. A high plus lens 48 is at the tip with a focal point of about 50 microns. Surrounding the fiberoptic in FIG. 7B is a suction conduit 42 through which the anterior capsule and central part of the lens are aspirated. In the alternative depicted in FIG. 7C the suction conduit 42 runs along one side of the long axis of the probe and a suction port 72 is provided near the tip of the probe. An irrigation line may be provided that is affixed to the long axis of the probe. In one embodiment, the system provides a volume of irrigation fluid that is balanced with the volume of material removed by the suction line.

Another fiberoptic constructed to remove the remaining toroid of lens tissue after the central lens core has been removed would be oriented with the lens and aspiration tip axis parallel to the fiberoptic axis. The instrument is manipulated on and in the lens. When femtosecond laser 44 emits energy it creates a lens ablation 50 microns deep. A low level vacuum is applied via the suction conduit 42 that acts as an aspiration port though which the lens fragments are aspirated. The high rate of laser firing and precise localization of the instrument and the remaining cataract enable precise lens removal with the capsule as the boundary to the process.

Although the seal disclosed herein has been described with some emphasis on use in cataract surgery, the seal may also be utilized in any other anterior segment ocular surgeries, such as glaucoma procedures where removal of trabecular meshwork is accomplished from an incision across the chamber and where it is desirable to maintain anterior segment stability. Furthermore, the seal disclosed herein may be utilized to seal incisions in other wall surfaces of the eye including for example the sclera for retinal or vitreal surgery.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

We claim:

1. A device for sealing a surgical incision in an ocular wall comprising:
a flexible bladder having an inner circumferential lip and an outer circumferential lip separated by a circumferential groove that are together dimensioned and adapted to sealably fit into a surgical incision in an ocular wall, the bladder further comprising at least one lumen dimensioned and adapted to conform to the contours of at least one instrument passed through the lumen into the eye while limiting egress of fluids from the eye.

2. The device of claim 1, further comprising an external reservoir in fluid communication with the flexible bladder and adapted to supply a fluid or gas to the bladder.

3. The device of claim 1, wherein the lips of the bladder comprise pliable internal rings.

4. The device of claim 1, wherein the flexible bladder is prefilled with a biologically compatible gas or fluid.

5. The device of claim 4, wherein the flexible bladder is a flexible solid polymer.

6. The device of claim 1, further comprising a pressure detector mounted on the bladder and adapted to monitor fluid pressure within the eye.

7. The device of claim 1, further comprising an infusion line mounted through a lumen in the bladder.

8. The device of claim 1, further comprising at least one irrigation line attached to the bladder adapted to pass fluids to and from the eye.

9. The device of claim 8, wherein the irrigation line is an infusion line.

10. An ocular seal comprising:
a flexible ring having a circumferential groove that is dimensioned and adapted to sealably fit into a surgical incision in an ocular wall, the ring further comprising at least one lumen dimensioned and adapted to conform to the contours of at least one instrument passed through the lumen into the eye while limiting egress of fluids from the eye.

11. The ocular seal of claim 10, wherein the flexible ring is prefilled with a fluid or gas.

12. The device of claim 10, wherein the flexible bladder is a flexible solid polymer.

13. The ocular seal of claim 10, wherein the ring is inflatable and further comprising an external reservoir in fluid communication with an interior of the inflatable ring.

14. The ocular seal of claim 10, further comprising an infusion line mounted through a lumen in the ring.

15. The device of claim 10, further comprising at least one irrigation line attached to the bladder and adapted to pass fluids to and from the eye.

16. The device of claim 15 wherein the irrigation line is an infusion line.

17. A system for surgery on an eye comprising:
a globe stabilization device that is adapted to circumferentially hug at least a portion of a limbus of the eye during surgery;
a lens ablation probe that includes a suction conduit; and
at least one ocular seal adapted to sealably fit into a surgical incision in an ocular wall and having a lumen adapted to conform to the contours of the lens ablation probe when the probe is passed through the lumen into the eye.

18. The system of claim 17, further comprising an anterior ocular coherence tomography or anterior segment ultrasound device set adapted and dimensioned for placement around the limbus to create a 3-dimensional image of the anterior segment during ocular surgery.

19. The system of claim 17, further comprising an infusion line mounted through the at least one ocular seal.

20. The system of claim 17, further comprising at least one irrigation line integral to at least one ocular seal and adapted to pass fluids to and from the eye.

21. The system of claim 20, wherein the irrigation line is an infusion line.

22. The system of claim 17, further comprising a pressure monitor mounted through at least one ocular seal.

23. The system of claim 17, wherein the lens ablation probe is a laser fiberoptic probe that includes a suction conduit, a fiber optic wave guide and at least one beam-manipulating device.

24. The system of claim 23, wherein the lens ablation probe further comprises an irrigation line.

25. The system of claim 23, wherein at least one beam-manipulating device is a prism, a mirror, a lens or any combination thereof

26. The system of claim 23, wherein the beam manipulating device is an angled laser delivery tip that directs laser energy at an angle of about 45° to about 135° to the fiber optic wave guide.
